(19) United States
(12) Patent Application Publication
(10) Pub. No.: US 2025/0003975 A1
(43) Pub. Date: Jan. 2, 2025

(54) Systems and Methods for Performing a Real-Time Glycan Assay of a Sample

(71) Applicant: AMGEN INC., Thousand Oaks, CA (US)

(72) Inventor: Chao-Hsiang WU, Thousand Oaks, CA (US)

(73) Assignee: AMGEN INC., Thousand Oaks, CA (US)

(21) Appl. No.: 18/663,909

(22) Filed: May 14, 2024

Related U.S. Application Data
(62) Division of application No. 16/635,938, filed on Jan. 31, 2020, now Pat. No. 12,025,618.
(60) Provisional application No. 62/539,798, filed on Aug. 1, 2017.

(51) Int. Cl. G01N 33/68; C12N 9/42; G01N 30/88

(52) U.S. Cl. CPC G01N 33/68; C12N 9/2437; G01N 30/88; G01N 2030/8804; G01N 2030/8836

(57) ABSTRACT

Systems and methods that facilitate the automatic (or substantially automatic) preparation of a sample of a product containing polypeptides for glycan analysis and automatic (or substantially automatic) performance of a glycan assay of that sample. Thus, the preparation and analysis can be performed substantially in-real time, or, in other words, much more quickly than presently allowed by conventional systems and methods.

### CROSS-REFERENCE TO RELATED APPLICATION

The present application claims the priority benefit of U.S. Provisional Patent Application No. 62/539,798, entitled “Systems and Methods for Performing a Real-Time Glycan Assay of a Sample” and filed Aug. 1, 2017, the entire disclosure of which is hereby incorporated by reference herein.

### FIELD OF THE DISCLOSURE

The present disclosure relates generally to glycan assays and, more specifically, to performing a real-time glycan assay of a sample.

### SEQUENCE LISTING

The present application is being filed with a sequence listing in electronic format. The sequence listing provided as a file titled, “51970_Seqlisting.txt” created Jul. 31, 2018 and is 263,980 bytes in size. The information in the electronic format of the sequence listing is incorporated herein by reference in its entirety.

### BACKGROUND

Assays are commonly performed to quantify one or more attributes of an analyte such as a drug, a biochemical substance, or a cell. An example of such an assay is the multi-attribute method (MAM) assay, which can detect and quantify Critical Quality Attributes (CQAs), identified by the Quality Target Product Profile (QTPP), of a sample (Development of a quantitative mass spectrometry multi-attribute method for characterization, quality control testing and disposition of biologics. Rogers R S, Nightlinger N S, Livingston B, Campbell P, Bailey R, Balland A. MAbs. 2015; 7(5): 881-90). The MAM assay is a manually-operated process that is performed in, for example, a Large Molecule Release Testing (LMRT) laboratory. MAM is a liquid chromatography (LC)—mass spectrometry (MS)-based peptide mapping method, having three steps: (1) sample preparation (which can include, for example, polypeptide denaturation, reduction, alkylation, and digestion); (2) separating the digested polypeptides by LC and detecting them by MS; and (3) analysis of the data for targeted CQAs and detection of new signal (i.e., peaks) when compared to a reference standard.

CQAs are chemical, physical, or biological properties that are present within a specific value or range values. For example, for large polypeptide therapeutic molecules, physical attributes and modifications of amino acids (the building blocks of polypeptides) are important CQAs that are monitored during and after manufacturing, as well as during drug development. Unlike conventional analytical assays that track changes in peak size and peak shape of whole or partial polypeptides, MAM detects specific CQAs at the amino acid level.

Analysis of the glycan profile of a polypeptide therapeutic is often a CQA.e.g. This is especially true in the case of biosimilar products, where the glycosylation profile of the biosimilar product must be comparable to that of the innovator product. Known processes for performing glycan assays require a sample of a product to be manually collected, delivered to a testing laboratory, and manually concentrated, purified,e.g. and prepared for analysis. The typical turnaround time for these known, manually operated, processes is about five days. This passage of time drives costs and delays in the development of drugs (innovator and biosimilar) and eventual drug release. For example, delays of five days accumulate during drug development, for example, when optimizing culture conditions for optimal glycosylation, impeding delivery of important and new polypeptide pharmaceuticals to patients. Furthermore, such delays result in profiles determined after manufacturing, resulting in re-manufacturing products that do not meet specifications as opposed to adjusting manufacturing parameters in real time. Thus, there is a need for efficient and faster methods to facilitate glycan analysis, including sample preparation for such analyses.

### SUMMARY

One aspect of the present disclosure provides a method for preparing a sample in real-time for glycan analysis. The method includes the steps of: (a) moving a sample comprising polypeptides to a polypeptide-binding column via a holding coil; (b) binding polypeptides in the sample to the polypeptide-binding column; (c) moving glycanases to the polypeptide-binding column via the holding coil to release glycans from the bound polypeptides; (d) moving a carrier solution to the polypeptide-binding column, via the holding coil, and through the polypeptide-binding column, thereby moving the released glycans out of the first polypeptide column; (c) mixing the released glycans with a glycan-labeling reagent downstream of the polypeptide-binding column; (f) moving the mixture of the released glycans and the glycan-labeling reagent to a reaction coil arranged downstream of the polypeptide-binding column; (g) incubating the mixture of the released glycans and the glycan-labeling reagent in the reaction coil, thereby labeling the glycans; (h) moving the mixture to a cooling coil arranged downstream of the reaction coil; (i) reducing a temperature of the mixture via the cooling coil; (j) moving the cooled mixture to a glycan-binding column arranged downstream of the cooling coil; (k) binding the labeled glycans to the glycan-binding column; and (l) moving an elution buffer to the glycan-binding column and through the glycan-binding column to elute the labeled glycans bound to the glycan-binding column.

Another aspect of the present disclosure provides a method for preparing a sample in real-time for glycan analysis in a closed system including a multi-port valve, a holding coil upstream of the multi-port valve, a polypeptide-binding column fluidly coupled to and downstream of a first port of the multi-port valve, a reaction coil arranged downstream of the polypeptide-binding column, a cooling coil arranged downstream of the reaction coil, and a glycan-binding column arranged downstream of the cooling coil and fluidly coupled to and downstream of a second port of the multi-port valve. The method includes: (a) moving, via a controller communicatively coupled to the closed system, a sample comprising polypeptides from a vessel containing the polypeptides to the holding coil; (b) positioning, via the controller, the multi-port valve in a first position in which the holding coil is fluidly coupled to the polypeptide-binding column via the first port of the multi-port valve, such that the sample flows to the polypeptide-binding column, whereby substantially all of the polypeptides in the sample bind to the first polypeptide column; (c) when the multi-port valve is in the first position, moving, via the controller, glycanases to the polypeptide-binding column via the holding coil to release glycans from the bound polypeptides, and then moving, via the controller, a carrier solution to the polypeptide-binding column, via the holding coil, and through the polypeptide-binding column, thereby moving the released glycans out of the polypeptide-binding column; (d) moving, via the controller, the released glycans toward the reaction coil; (c) moving, via the controller, a glycan labeling reagent toward the released glycans prior to reaching the reaction coil, such that the glycan labeling reagent mixes with the released glycans and the enzymes; (f) moving, via the controller, the mixture to the reaction coil, whereby the glycans in the mixture are labeled; (g) moving, via the controller, the mixture to a cooling coil arranged downstream of the reaction coil, thereby reducing a temperature of the mixture; (h) moving, via the controller, the cooled mixture to a glycan column arranged downstream of the cooling coil, whereby substantially all of the labeled glycans bind to the glycan-binding column; (i) positioning, via the controller, the multi-port valve in a second position in which the glycan-binding column is fluidly coupled to a source of elution buffer solution via the second port of the multi-port valve; (j) when the multi-port valve is in the second position, moving, via the controller, the elution buffer from the elution buffer source to the glycan-binding column, and through the glycan-binding column, thereby eluting glycans bound to the glycan-binding column; and (k) moving, via the controller, the eluted glycans to a glycan analysis device.

Another aspect of the present disclosure provides a closed system for preparing a sample in real-time for glycan analysis. The closed system includes a multi-port valve, a holding coil upstream of the multi-port valve, a polypeptide-binding column fluidly coupled to and downstream of a first port of the multi-port valve, a reaction coil arranged downstream of the polypeptide-binding column, a cooling coil arranged downstream of the reaction coil, a glycan-binding column fluidly coupled to and downstream of the cooling coil, the glycan-binding column fluidly coupled to the multi-port valve via a second port of the multi-port valve, and a controller communicatively coupled to the multi-port valve. The controller includes a memory, a processor, and logic stored on the memory and executable by the processor to: (a) move a sample of a product containing polypeptides to the holding coil; (b) position the multi-port valve in a first position in which the holding coil is fluidly coupled to the polypeptide-binding column via the first port of the multi-port valve, such that the sample flows to the first column, whereby substantially all of the polypeptides in the sample bind to the polypeptide-binding column; (c) when the multi-port valve is in the first position, move glucanases to the polypeptide-binding column via the holding coil to release glycans from the bound polypeptides, and then move a carrier solution to the polypeptide-binding column, via the holding coil, and through the polypeptide-binding column, thereby moving substantially all of the released glycans out of the polypeptide-binding column; (d) move the released glycans toward the reaction coil; (e) move a glycan-labeling reagent toward the released glycans prior to reaching the reaction coil, such that the glycan-labeling reagent mixes with the released glycans; (f) move the mixture of the released glycans and the glycan-labeling reagent to the reaction coil, whereby the glycans in the mixture are labeled; (g) move the mixture to a cooling coil arranged downstream of the reaction coil, wherein the cooling coil reduces a temperature of the mixture; (h) move the mixture to the glycan-binding column, whereby the labeled glycans bind to the glycan-binding column; (i) position the multi-port valve in a second position in which the glycan-binding column is fluidly coupled to a source of elution buffer solution via the second port of the multi-port valve; and (j) when the multi-port valve is in the second position, move an elution buffer solution from the elution buffer solution source to the glycan-binding column, and through the glycan-binding column, thereby eluting glycans bound to the glycan-binding column.

### BRIEF DESCRIPTION OF THE DRAWINGS

FIG. 1 is a schematic diagram of a system for performing an online, real-time glycan assay assembled in accordance with the teachings of the present disclosure.

FIG. 2 is a schematic diagram of a controller of the system illustrated in FIG. 1.

FIGS. 3A and 3B are graphs depicting the results of a study monitoring the effectiveness of the system of FIG. 1 over a production run of 32 days.

FIG. 3C is a graph depicting a snapshot of the results of FIG. 3A over a four-day period of time.

FIGS. 3D and 3E are additional graphs depicting the results of the study.

### DETAILED DESCRIPTION OF THE PREFERRED EMBODIMENTS

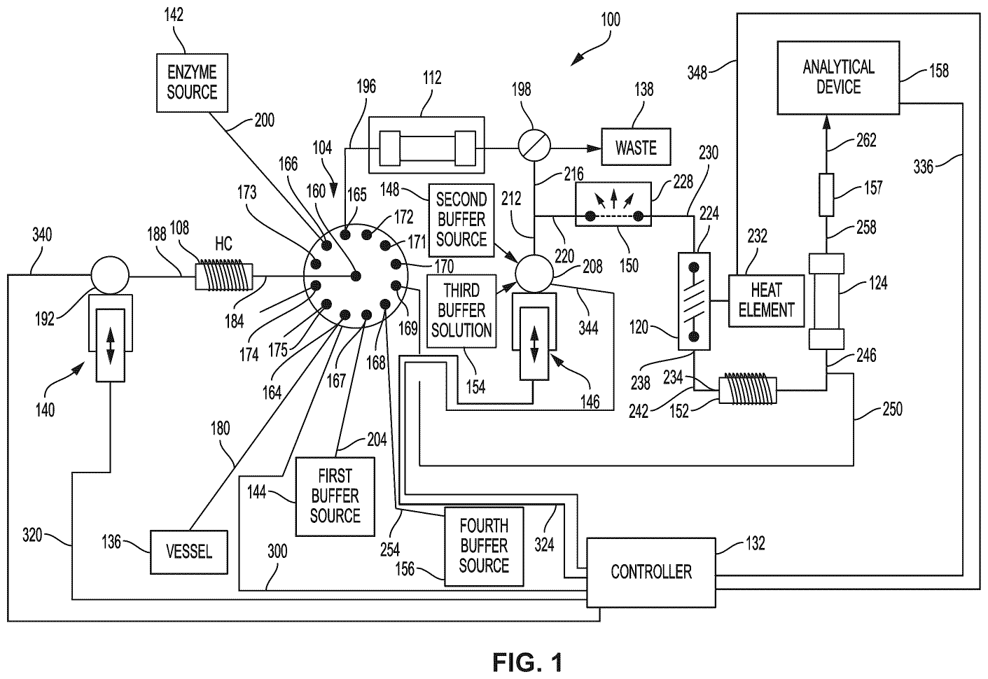

FIG. 1 illustrates a schematic diagram of a system 100 assembled in accordance with the teachings of the present disclosure. The system 100, which can be located at or in a laboratory (e.g., a Large Molecule Release Testing laboratory) is a closed system for automatically or substantially automatically preparing a sample of a product containing polypeptides for glycan analysis and automatically performing a glycan assay of that sample, as is described in greater detail below. By automating (or substantially automating) this process using the system 100, the assay can be performed in real-time (or substantially in real-time), such that the entire process can be performed, and the desired result obtained, in a matter of hours (e.g., 2 to 3 hours), a significant improvement over the 5 days typically required by the conventionally known, manually-operated processes. Moreover, the closed nature of the process that utilizes the system 100 maintains sterile conditions.

In this version, the polypeptides are therapeutic polypeptides. Therapeutic polypeptides are discussed below.

The system 100 illustrated in FIG. 1 generally includes a multi-port valve 104, a holding coil 108, a first column 112, a reaction coil 120, a second column 124, and a controller 132. In the version illustrated in FIG. 1, the system 100 also includes a vessel 136, a waste chamber 138, a first pump 140, an enzyme source 142, a first buffer source 144, a second pump 146, a second buffer source 148, a vacuum apparatus 150, a cooling coil 152, a third buffer source 154, a fourth buffer source 156, a mixing chamber 157, and an analytical device 158 for analyzing the glycans. In other versions, however, the system 100 may not include one or more of these components. As an example, the system 100 may not include the vessel 136, the vacuum apparatus 150, the cooling coil 152, and/or the analytical device 158. In any event, generally conventional plumbing extends between each of the components of the system 100 so as to facilitate fluid communication between components of the system 100 when desired, as is described in greater detail below.

The multi-port valve 104 is generally configured to control fluid communication between the various components of the system 100. In this version, the multi-port valve 104 is a twelve satellite port and a central shared port valve. In other words, the multi-port valve 104 has a central port 160 and twelve satellite ports 164-175 that are selectively fluidly coupled to the central port 160. The multi-port valve 104 is movable between twelve different positions that fluidly couple the central port 160 with the twelve different satellite ports 164-175, respectively (some of which are not utilized in the operation of the system 100 of FIG. 1). In other versions, the multi-port valve 104 can have more or less satellite ports, can be a different type of valve, or can be replaced by one or more different valves (each having one or more ports). As an example, the multi-port valve 104 can be replaced by a plurality of single port valves separately connected to and controlled by the controller 132, with each of the single port valves effectively replacing one of the satellite ports 164-175.

The vessel 136 is generally configured to hold or store the product containing polypeptides having glycans that are to be prepared and analyzed. The vessel 136 in this version takes the form of a bioreactor that holds or stores the product. In other versions, however, the vessel 136 can instead take the form of a cell culture vessel, such as a flask, a plate, etc. The vessel 136 is fluidly coupled to the satellite port 164 of the valve 104 via a conduit 180 of the plumbing, such that the valve 104 can, when desired, obtain a sample of the product contained in the vessel 136 from the vessel 136.

The holding coil 108 is located upstream of the valve 104 and is fluidly coupled to the central port 160 of the valve 104 via a conduit 184 of the plumbing. The holding coil 108 is thus arranged to receive the sample of the product from the vessel 136, via the valve 104, when the valve 104 is in a first position in which the central port 160 is fluidly coupled to the satellite port 164.

The first pump 140 is located upstream of and fluidly coupled to the holding coil 108 via a conduit 188 of the plumbing. The pump 140 in this version takes the form of a syringe pump that is generally configured to help obtain various materials and help move those materials to and between various components of the system 100, as is described in greater detail below. In other versions, the pump 140 can be a different type of pump and/or multiple pumps 140 can be used for different materials and/or to output materials to different components.

A valve 192 is located between the first pump 140 and the holding coil 108 to selectively fluidly couple the first pump 140 to the holding coil 108. More particularly, the valve 192 is movable between a first position, in which the pump 140 is fluidly coupled to the holding coil 108, and a second position, in which the pump 140 is fluidly isolated from the holding coil 108. In other words, the pump 140 is selectively fluidly coupled to the holding coil 108 depending upon the position of the valve 192.

When desired, and when the valve 104 is in the first position (in which the central port 160 is fluidly coupled to the satellite port 164) and the valve 192 is in the first position, the first pump 140 can facilitate movement of the sample of the product from the vessel 136 to the holding coil 108, via the valve 104. The sample of the product can in turn be passed to the first column 112 for binding.

The first column 112 is located downstream of the valve 104 and has an inlet fluidly coupled to the satellite port 165 of the valve 104 via a conduit 196 of the plumbing. Thus, when the valve 104 is in a second position in which the central port 160 is fluidly coupled to the satellite port 165, the first pump 140 can help move (e.g., pump, pass) the sample of the product from the holding coil 108 to the first column 112, via the valve 104. The first column 112 has an outlet that is fluidly coupled to a three-way valve 198 configured to selectively fluidly connect the first column 112 with either the waste chamber 138 or the reaction coil 120. More particularly, the three-way valve 198 is movable between a first position, in which the first column 112 is fluidly coupled to the waste chamber 138 and fluidly isolated from the vacuum apparatus 150, and a second position, in which the first column 112 is fluidly coupled to the vacuum apparatus 150 and fluidly isolated from the waste chamber 138. In this version, the first position of the three-way valve 198 is the default position, such that the three-way valve 198 only moves from the first position to the second responsive to actuation of the three-way valve 198 (e.g., by the controller 132, by applying a current to the valve 198). In other versions, however, the second position of the three-way valve 198 may be the default position. In any case, when the first column 112 receives the sample, the first column 112 is configured to bind substantially all of the polypeptides from the sample as the sample flows therethrough, and the three-way valve 198 is in its first position. In this manner, the first column 112 substantially separates the polypeptides in the sample from a remainder of the sample, which can be passed to the waste chamber 138 via the three-way valve 198.

The first column 112, which can also be referred to herein as a polypeptide-binding column, is selected from a group consisting of a protein A column, a protein G column, a protein A/G column, a protein L column, an amino acid column, an avidin column, a streptavidin column, a carbohydrate bonding column, a carbohydrate column, a glutathione column, a heparin column, a hydrophobic interaction column, an immunoaffinity column, a nucleotide/coenzyme column, a specialty column, and an immobilized-metal affinity chromatography (IMAC) column. For example, in the case of polypeptides that are human IgGs of subclasses 1, 2, or 4, IgM, IgA, or IgE (and comprising a human Fc portion and/or a Fab region of the human VH3 family), protein A columns are useful. Protein G can be used to purify human IgGs of subclasses 1-4. Recombinant fusion protein A/G can also be used to purify all of these classes of human antibodies, as the fusion protein provides protein A and protein G binding sites. Thus, protein A/G fusion proteins can be used to purify human IgG, IgA, IgE, and IgM. Furthermore, protein L can be used to purify human IgG, IgM, IgA, IgE and IgD, provided the target antibodies have an appropriate kappa (κ) subtype light chain (i.e., VκI, VκIII and VκIV subtypes); protein L can also be used to purify Fab and scFv fragments also having the appropriate κ chain subtype, as protein L binds the variable (V) chain of antibodies.

After substantially all of the polypeptides from the sample are bound to the first column 112, a solution containing (e.g., consisting of) glucanases is supplied to the system 100 by the enzyme source 142, which is fluidly coupled to the satellite port 166 of the valve 104 via a conduit 200 of the plumbing. In this version, the solution containing the glucanases is moved to the holding coil 108, via the valve 104 and with assistance from the pump 140, when the valve 104 is in a third position in which the central port 160 is fluidly coupled to the satellite port 166 and when the valve 192 is in the first position. In turn, the solution containing the glucanases is moved from the holding coil 108 to the first column 112, via the valve 104 and again with assistance from the pump 140, when the valve 104 is in the second position (in which the central port 160 is fluidly coupled to the satellite port 165) and when the valve 192 is in the first position. In other versions, however, the solution containing the glucanases can be provided to the first column 112 in a different manner (e.g., directly to the first column 112, without assistance from the pump 140). In any event, when the solution containing the glucanases reaches the first column 112, the glucanses in the solution infuse the polypeptides bound to the first column 112. Infusion is generally facilitated by incubating the first column 112, which is, in this version, accomplished by maintaining the first column 112 at a temperature of about 35° C. to about 40° C., and, more preferably, a temperature of about 37° C. To this end, a heating element (e.g., a heating coil, an induction heater, a heat pump, a cartridge heater) can be connected to the first column 112 to provide the heat necessary to maintain the first column 112 at the desired temperature. In any case, it is appreciated that infusion of the glucanses in the solution serves to release glycans in the bound polypeptides from the bound polypeptides into the solution.

The glucanases in the solution are preferably selected from the group consisting of endoglycosidases, glycosamidases, and O-glycanases, and combinations thereof. When the glucanases are or include endoglycosidases, the endoglycosidases are preferably selected from the group consisting of endoglycosidase D, endoglycosidase F (endoglycosidase F1, endoglycosidase F2, and endoglycosidase F3 and combinations thereof), endoglycosidase H, endoglycosidase S, endoglycosidase M, and endoglycosidase B. When the glucanases are or include glycosamidases, the glycosamidases are preferably selected from the group consisting of glycopeptidases, peptide N-glycosidases, PNGases, N-glycohydrolases, and N-glycanases. When the glucanases are or include PNGases, the PNGases preferably include peptide: N-glycosidase F (PNGF). When the glucanases are or include O-glycanases, the O-glycanases preferably are endo-GalNAc-ase D or endoGalNAc-ase A.

After glycans in the bound polypeptides are released (or substantially released) into the solution, and with the three-way valve 198 in (or moved) to its second position, a carrier solution (e.g., de-ionized (DI) water) is moved to and through the first column 112 so as to move the released glycans (in solution with the glucanases) out of the first column 112. The carrier solution is generally supplied to the system 100 by the first buffer source 144, which is fluidly coupled to the satellite port 167 of the valve 104 via a conduit 204 of the plumbing. In this version, the carrier solution is moved to the holding coil 108, via the valve 104 and with assistance from the pump 140, when the valve 104 is in a fourth position in which the central port 160 is fluidly coupled to the satellite port 167 and when the valve 192 is in the first position. In turn, the carrier solution is moved from the holding coil 108 to and through the first column 112, via the valve 104 and again with assistance from the pump 140, when the valve 104 is in the second position (in which the central port 160 is fluidly coupled to the satellite port 165) and when the valve 192 is in the first position. In other versions, however, the carrier solution can be provided to the first column 112 in a different manner (e.g., directly to the first column 112 and/or without assistance from the pump 140). In any event, it is appreciated that the carrier solution carries or moves the released glycans (and the glucanases) through and out of the first column 112.

The second pump 146 is located downstream of the first column 112. Like the first pump 140, the second pump 146 in this version takes the form of a syringe pump that is generally configured to help obtain various materials and help move those materials to and between various components of the system 100, as is described in greater detail below. In other versions, the pump 146 can be a different type of pump and/or multiple pumps 146 can be used for different materials and/or to output materials to different components.

A valve 208 is located between the second pump 146 and a conduit 212 of the plumbing located downstream of the first column 112 to selectively fluidly couple the second pump 146 to the conduit 212 downstream of the first column 112. More particularly, the valve 208 is movable between a first position, in which the pump 140 is fluidly coupled to the holding coil 108, and a second position, in which the pump 140 is fluidly isolated from the holding coil 108. In other words, the pump 140 is selectively fluidly coupled to the holding coil 108 depending upon the position of the valve 192.

The second pump 146 in this version is also fluidly coupled to the second buffer source 148, which is configured to supply a glycan labeling reagent such as, for example, a flurophore (e.g., selected from the group consisting of 2-aminobenzoic acid, 8-aminonaphthalene-1,3,6-trisulfonic acid disodium salt, 8-aminonaphthalene-1,3,6-trisulfonic acid trisodium salt, and anthranilamide, 4-methoxybenzamidine) or a chromophore (e.g., 3-methyl-1-phenyl-2-pyrazoline-5-one or phenylhydrazine). Thus, when the valve 208 is in the first position, the second pump 146 can move (e.g., pump, direct) the glycan labeling reagent through the conduit 212 and toward the released glycans carried from the first column 112 via a conduit 216 of the plumbing. The glycan labeling reagent thus mixes with the released glycans at a position downstream of the first column 112.

The vacuum apparatus 150 is located downstream of both the first column 112 and the second pump 146. The vacuum apparatus 150 is fluidly coupled to both the conduit 216 (carrying the released glycans from the first column 112) and the conduit 212 (carrying the glycan reagent from the second pump 146) via a conduit 220 of the plumbing. The vacuum apparatus 150 is therefore arranged to receive the mixture of the released glycans and the glycan reagent. As the mixture flows through the vacuum apparatus 150, the vacuum apparatus 150 removes gas (e.g., air) bubbles from the mixture (i.e., degasses the mixture), which, as is known in the art, can inhibit performance of the downstream components of the system 100 (by, for example, interfering with gradient delivery).

The reaction coil 120 is located downstream of the vacuum apparatus 150 (and, thus, the first column 112 and the second pump 146 as well). An inlet 224 of the reaction coil 120 is fluidly coupled to an outlet 228 of the vacuum apparatus 150 via a conduit 230 of the plumbing, such that the reaction coil 120 is arranged to receive the mixture including the glycan labeling reagent and the released glycans. The reaction coil 120 is, in turn, generally configured to facilitate labeling (e.g., fluorescently labeling) of the released glycans. This is generally accomplished by incubating the mixture in the reaction coil 120. To this end, the reaction coil 120 is, in this version, heated by a heating clement 232 connected to (e.g., positioned immediately adjacent, surrounding) the reaction coil 120. In other words, the heating element 232 can apply heat, preferably heat having a temperature of about 75° C. to about 85° C., and, more preferably, heat having a temperature of about 80° C., to the reaction coil 120 to encourage labeling. The heating element 232 can, for example, take the form of a heating coil, an induction heater, a heat pump, a cartridge heater, an electrical resistance wire, or other element suitable for heating one or more portions of the reaction coil 120.

The cooling coil 152, which in this version is made of stainless steel, is located downstream of and fluidly coupled to the reaction coil 120. More particularly, an inlet 234 of the cooling coil 152 is fluidly coupled to an outlet 238 of the reaction coil 120 via a conduit 242 of the plumbing, such that the cooling coil 152 is arranged to receive the mixture including the glycan labeling reagent and the released (and now labeled) glycans. The cooling coil 152 is maintained (e.g., by the controller 132) at a temperature less than the temperature at which the reaction coil 120 is maintained. The cooling coil 152 thus not only reduces the temperature of the mixture, but also stretches out the mixture as it flows therethrough, thereby reducing the organic content of the mixture. In doing so, the reaction coil 120 enhances the ability of the glycans to bind to the second column.

The second column 124, also referred to herein as the glycan-binding column 124, preferably takes the form of a porous graphitic carbon column that is located downstream of and fluidly coupled to the cooling coil 152 via a conduit 246. The second column 124 is thus arranged to receive the cooled and diluted mixture from the cooling coil 152. To facilitate movement of the cooled and diluted mixture from the cooling coil 152 to the second column 124, the second pump 146 can, when the valve 208 is in the first position, move a buffer solution (e.g., formed of acetonitrile (5%) with 0.1% TFA solution) from the third buffer source 154, which is fluidly coupled thereto to the second column 124 via the conduits 212, 220, 230, 234, and 246. In any event, when the second column 124 receives the mixture from the cooling coil 152, the second column 124 is configured to bind the labeled glycans from the mixture as the mixture flows therethrough. In this manner, the second column 124 separates the labeled glycans in the mixture from a remainder of the mixture, which can be passed to waste (not shown).

The second column 124 is also fluidly coupled to the satellite port 169 of the valve 104 via a conduit 250 of the plumbing. In this version, the conduit 250 joins or intersects the conduit 246 upstream of the second column 124, but in other versions, the conduit 250 can instead bypass the conduit 246 and be directly connected to the second column 124. In any case, an elution buffer solution (e.g., formed of acetonitrile (5%) with 0.1% TFA solution) is moved to and through the second column 124 via the conduit 250 so as to clute substantially all of the bound and labeled glycans. The elution buffer solution is generally supplied by the fourth buffer source 156, which is fluidly coupled to the satellite port 168 of the valve 104 via a conduit 254 of the plumbing. In this version, the elution buffer solution is moved from the fourth buffer source 156 to the holding coil 108, via the valve 104 and with assistance from the pump 140, when the valve 104 is in a fifth position in which the central port 160 is fluidly coupled to the satellite port 168 and when the valve 192 is in the first position. In turn, the elution buffer solution is moved from the holding coil 108 to and through the second column 124, via the valve 104 and again with assistance from the pump 140, when the valve 104 is in a sixth position in which the central port 160 is fluidly coupled to the satellite port 169 and when the valve 192 is in the first position. In other versions, however, the elution buffer solution can be provided to the second column 124 in a different manner (e.g., directly to the second column 124 and/or without assistance from the pump 140). In any event, the supplied elution buffer solution substantially elutes the glycans bound to the second column 124 and carries or moves those eluted glycans through and out of the second column 124.

In this version, the elution buffer solution carries the glycans from the second column 124 to the mixing chamber 157, which is located downstream of and fluidly coupled to the second column 124 via a conduit 258 of the plumbing. The mixture of the elution buffer solution and the glycans flows through the mixing chamber 157, which helps to reduce the organic content of the mixture and serves to adjust the elution buffer component of the glycans to match the starting chromatographic mobile phase condition. In other words, the mixing chamber 157 helps to ensure that the mixture is representative of the original sample obtained from the vessel 136.

After the mixture has been in the mixing chamber 157 for the pre-determined amount of time, the mixture can be moved, e.g., using the elution buffer solution, to the analytical device 158, which can, for example, take the form of a liquid chromatography device, a high-performance liquid chromatography device, an ultra high-performance liquid chromatography device, a mass spectrometry device, a glycan analysis device, another analysis device, or a combination thereof. In this version, the analytical device 158 is located downstream of the second column 124 and is fluidly coupled to the second column 124 via a conduit 262 of the plumbing. Thus, in this version, the mixture can be automatically moved to the analytical device 158 for glycan analysis (e.g., for quantitating and separating the glycans in the mixture). In other versions, however, the analytical device 158 can not be part of the system 100 (e.g., not fluidly coupled to the third column 128), in which case the mixture can be moved to the analytical device 158 in a different manner (e.g., manually).

As briefly noted above, the system 100 also includes the controller 132, which in this version is communicatively coupled or connected to various components of the system 100 to monitor and facilitate or direct the above-described operation of the system 100 by transmitting signals (e.g., control signals, data) to and receiving signals (e.g., data) from the various components of the system 100. The controller 132 can be located immediately adjacent the other components of the system 100 (e.g., in the same environment as the system 100) or can be remotely located from the other components of the system 100. As illustrated, the controller 132 is communicatively coupled or connected to the multi-port valve 104 via a communication network 300, the first and second pumps 140, 146 via communication networks 320, 324, respectively,, the analytical device 158 via a communication network 336, the valves 192, 208 via communication networks 340, 344, respectively, and the heat element 232 via a communication network 348. In other versions, the controller 132 can be communicatively coupled or connected to more or less components of the system 100, e.g., the holding coil 108, the first column 112, the reaction coil 120, the second column 124, the vessel 136, the cooling coil 152, the mixing chamber 157, and/or the three-way valve 198.

As used herein, the phrases “communicatively coupled” and “connected” are defined to mean directly coupled or connected to or indirectly coupled or connected through one or more intermediate components. Such intermediate components can include hardware and/or software-based components. It is appreciated that the networks 300-348 can be wireless networks, wired networks, or combinations of a wired and a wireless network (e.g., a cellular telephone network and/or 802.11x compliant network), and can include a publicly accessible network, such as the Internet, a private network, or a combination thereof. The type and configuration of the networks 300-348 is implementation dependent, and any type of communications networks which facilitate the described communications between the controller 132 and the components of the system 100, available now or later developed, can be used.

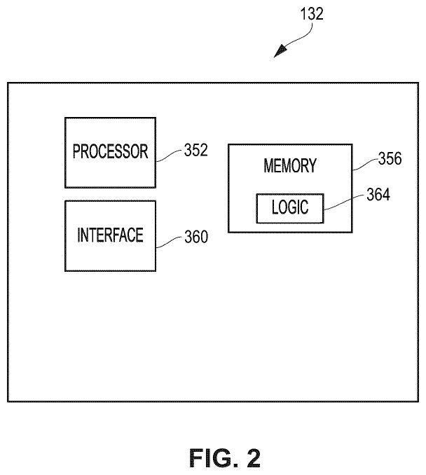

As shown in FIG. 2, the controller 132 includes a processor 352, a memory 356, a communications interface 360, and computing logic 364. The processor 352 can be a general processor, a digital signal processor, ASIC, field programmable gate array, graphics processing unit, analog circuit, digital circuit, or any other known or later developed processor. The processor 352 operates pursuant to instructions in the memory 356. The memory 356 can be a volatile memory or a non-volatile memory. The memory 356 can include one or more of a read-only memory (ROM), random-access memory (RAM), a flash memory, an electronic erasable program read-only memory (EEPROM), or other type of memory. The memory 356 can include an optical, magnetic (hard drive), or any other form of data storage device.

The communications interface 360 is provided to enable or facilitate electronic communication between the controller 132 and the components of the refrigeration system 100 via the networks 300-348. The communications interface 360 can be or include, for example, one or more universal serial bus (USB) ports, one or more Ethernet ports, and/or one or more other ports or interfaces. The electronic communication can occur via any known communications protocol, including, by way of example, USB, RS-232, RS-485, WiFi, Bluetooth, and/or any other suitable communications protocol.

The logic 364 generally includes one or more control routines and/or one or more sub-routines embodied as computer-readable instructions stored on the memory 356. The control routines and/or sub-routines can perform PID (proportional-integral-derivative), fuzzy logic, nonlinear, or any other suitable type of control. The processor 352 generally executes the logic 364 to perform actions related to the operation of the system 100.

Generally speaking, the logic 364, when executed, causes the processor 352 to control components of the system 100, particularly the multi-port valve 104, the first and second pumps 140, 146, the analytical device 158, the first and second valves 192, 208, and the heating element 232, such that the system 100 operates in the desired manner discussed herein. More particularly, the logic 364 can, when executed, cause the processor 352 to (i) move the multi-port valve 104 to or between any of the positions described herein, thereby fluidly coupling various components of the system 100 as described above, (ii) control the first pump 140 (e.g., cause the pump 140 to obtain or direct materials as described above, (iii) control the second pump 146 (e.g., cause the pump 146 to obtain or direct materials as described above), (iv) open or close the valve 192, (v) open or close the valve 208, (vi) control the heating element 232 (when employed) to control the temperature of the reaction coil 120 by selectively applying heat to the reaction coil 120, and (vii) control the analytical device 158.

In other versions, the logic 364 can, when executed by the processor 352, cause additional, less, and/or different functionality to be performed. As an example, the logic 364, when executed by the processor 352, can not move the glycans from the second column 124 to the analytical device 158 or cause the analytical device 156 to perform the desired analysis. Moreover, in other versions, the logic 364 can be executed by the processor 352 in a different order than described herein. Finally, it is appreciated that the logic 364 can be executed by the processor 352 any number of different times, as the system 100 can be used to perform real-time analyses of multiple samples (from the same product and/or from a different product).

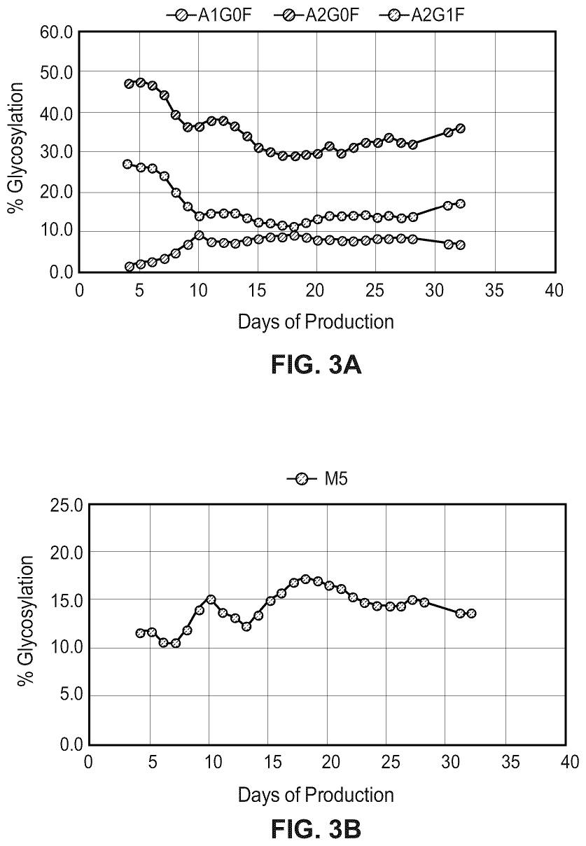

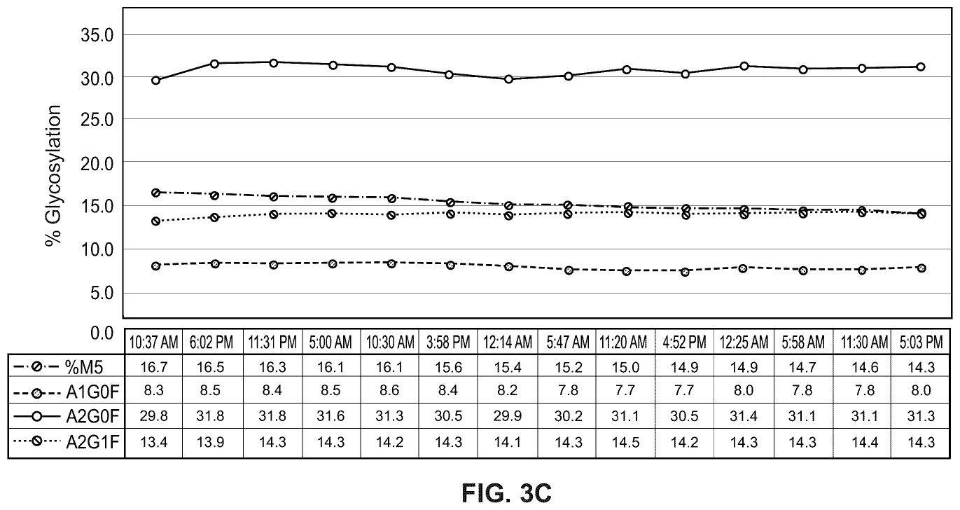

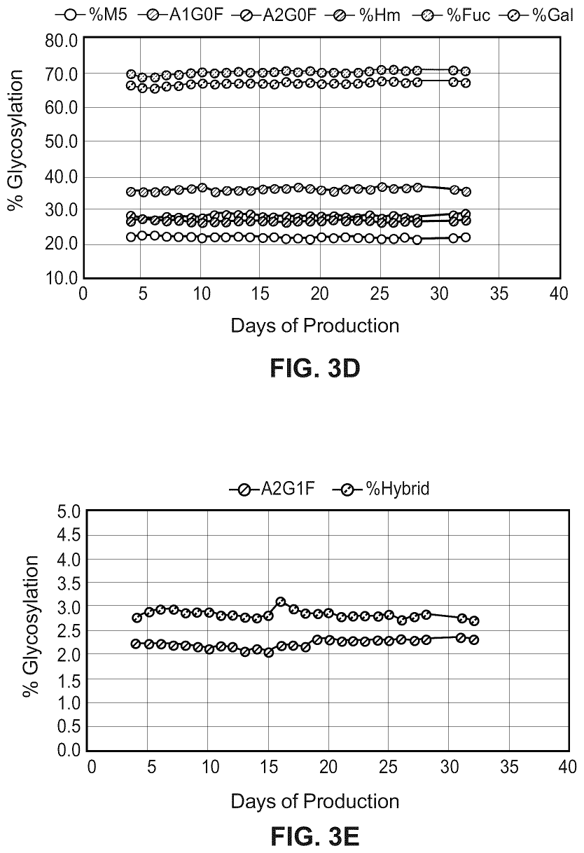

FIGS. 3A-3E illustrate the results of an online and real-time glycosylation profile study for monitoring the effectiveness of the system 100 in preparing a sample of an antibody-based therapeutic polypeptide. In particular, the study monitored the effectiveness of the system 100 over a production run of 32 days. The study began monitoring and collecting CQA data such as % A1G0F, % A2G0F, % A2G1F (glycans), and % M5 (mannose 5) on day four of the 32-day production run. As illustrated in FIGS. 3A and 3B, the system 100 capably and effectively performed the intended functionality discussed herein over the entire duration of the 32-day production run, and as illustrated in FIGS. 3D and 3E, the CQA data collected between day four and day 32 was consistent, i.e., there was no change in product quality over time, thereby demonstrating the effectiveness and robustness of the system 100. This was in spite of the fact that on day 20 of the production run, oxygen gas flow to the vessel 136 was interrupted for two hours due to a leak in the oxygen tank. In turn, the sampling frequency from the vessel 136 was increased (in this case from once every 24 hours to once every six hours) in order to monitor the vessel 136 at a higher resolution and assess the risk of continuing the production run. However, as demonstrated in FIGS. 3A-3C, this interruption only temporarily affected some of the CQA data and the CQA data quickly returned to pre-interruption values and stabilized. Accordingly, the decision was made to continue the production run.

### Therapeutic Polypeptides

Proteins, including those that bind to one or more of the following, can be useful in the disclosed devices and methods. These include CD proteins, including CD3, CD4, CD8, CD19, CD20, CD22, CD30, and CD34; including those that interfere with receptor binding. HER receptor family proteins, including HER2, HER3, HER4, and the EGF receptor. Cell adhesion molecules, for example, LFA-I, MoI, pl50, 95, VLA-4, ICAM-I, VCAM, and alpha v/beta 3 integrin. Growth factors, such as vascular endothelial growth factor (“VEGF”), growth hormone, thyroid stimulating hormone, follicle stimulating hormone, luteinizing hormone, growth hormone releasing factor, parathyroid hormone, Mullerian-inhibiting substance, human macrophage inflammatory protein (MIP-I-alpha), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors. Insulins and insulin-related proteins, including insulin, insulin A-chain, insulin B-chain, proinsulin, and insulin-like growth factor binding proteins. Coagulation and coagulation-related proteins, such as, among others, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-I to IL-33 and IL-I to IL-33 receptors, such as the IL-8 receptor, among others. Viral antigens, including an AIDS envelope viral antigen. Lipoproteins, calcitonin, glucagon, atrial natriuretic factor, lung surfactant, tumor necrosis factor-alpha and-beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies. Myostatins, TALL proteins, including TALL-I, amyloid proteins, including but not limited to amyloid-beta proteins, thymic stromal lymphopoietins (“TSLP”), RANK ligand (“OPGL”), c-kit, TNF receptors, including TNF Receptor Type 1, TRAIL-R2, angiopoictins, and biologically active fragments or analogs or variants of any of the foregoing.

Exemplary polypeptides and antibodies include Activase® (Alteplase); alirocumab, Aranesp® (Darbepoetin-alfa), Epogen® (Epoctin alfa, or erythropoietin); Avonex® (Interferon β-Ia); Bexxar® (Tositumomab); Betaseron® (Interferon-β); bococizumab (anti-PCSK9 monoclonal antibody designated as L1L3, sec U.S. Pat. No. 8,080,243); Campath® (Alemtuzumab); Dynepo® (Epoetin delta); Velcade® (bortezomib); MLN0002 (anti-α4β7 mAb); MLN1202 (anti-CCR2 chemokine receptor mAb); Enbrel® (etanercept); Eprex® (Epoctin alfa); Erbitux® (Cetuximab); evolocumab; Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-cpoctin beta); Mylotarg® (Gemtuzumab ozogamicin); Raptiva® (efalizumab); Cimzia® (certolizumab pegol); Soliris™ (Eculizumab); Pexelizumab (Anti-C5 Complement); MEDI-524 (Numax®); Lucentis® (Ranibizumab); Edrecolomab (,Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMI); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 146B7-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ETI211 (anti-MRSA mAb), IL-I Trap (the Fc portion of human IgG1 and the extracellular domains of both IL-I receptor components (the Type I receptor and receptor accessory protein)), VEGF Trap (Ig domains of VEGFR1 fused to IgG1 Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi™ (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-I) and MDX-1388); anti-CD22 dsFv-PE38 conjugates (CAT-3888 and CAT-8015); anti-CD25 mAb (HuMax-TAC); anti-TSLP antibodies; anti-TSLP receptor antibody (U.S. Pat. No. 8,101,182); anti-TSLP antibody designated as A5 (U.S. Pat. No. 7,982,016); (anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-cotaxinl mAb (CAT-213); anti-FGF8 mAb; anti-ganglioside GD2 mAb; anti-sclerostin antibodies (sec, U.S. Pat. Nos. 8,715,663 or 7,592,429) anti-sclerostin antibody designated as Ab-5 (U.S. Pat. Nos. 8,715,663 or 7,592,429); anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AIN457); anti-IL2Ra mAb (HuMax-TAC); anti-IL5 Receptor mAb; anti-integrin receptors mAb (MDX-Ol8, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PDImAb (MDX-1 106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); and an amyloid-beta monoclonal antibody comprising sequences, SEQ ID NO:8 and SEQ ID NO:6 (U.S. Pat. No. 7,906,625).

Examples of antibodies suitable for the methods and pharmaceutical formulations include the antibodies shown in Table 1. Other examples of suitable antibodies include infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, asclizumab, altinumab, atlizumab, atorolimiumab, tocilizumab, bapincuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, cculizumab, edobacomab, edrecolomab, efalizumab, efungumab, clotuzumab, elsilimomab, enavatuzumab, enlimomab pegol, enokizumab, enoticumab, ensituximab, epitumomab cituxetan, epratuzumab, erenumab, erlizumab, ertumaxomab, ctaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixckizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanczumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox.

Antibodies also include adalimumab, bevacizumab, blinatumomab, cetuximab, conatumumab, denosumab, eculizumab, erenumab, evolocumab, infliximab, natalizumab, panitumumab, rilotumumab, rituximab, romosozumab, tezepelumab, and trastuzumab, and antibodies selected from Table 1.

| | | | HC | | | LC | HC |
|---|---|---|---|---|---|---|---|
| Target | | Viscos- | Type | | | SEQ | SEQ |
| (informal | Conc. | ity | (including | LC | | ID | ID |
| name) | (mg/ml) | (cP) | allotypes) | Type | pI | NO | NO |
| anti- | 142.2 | 5.0 | IgG1 (f) | Kappa | 9.0 | 1 | 2 |
| amyloid | | | (R; EM) | | | | |
| GMCSF | 139.7 | 5.6 | IgG2 | Kappa | 8.7 | 3 | 4 |
| (247) | | | | | | | |
| CGRPR | 136.6 | 6.3 | IgG2 | Lambda | 8.6 | 5 | 6 |
| RANKL | 152.7 | 6.6 | IgG2 | Kappa | 8.6 | 7 | 8 |
| Sclerostin | 145.0 | 6.7 | IgG2 | Kappa | 6.6 | 9 | 10 |
| (27H6) | | | | | | | |
| IL-1R1 | 153.9 | 6.7 | IgG2 | Kappa | 7.4 | 11 | 12 |
| Myostatin | 141.0 | 6.8 | IgG1 (z) | Kappa | 8.7 | 13 | 14 |
| | | | (K; EM) | | | | |
| B7RP1 | 137.5 | 7.7 | IgG2 | Kappa | 7.7 | 15 | 16 |
| Amyloid | 140.6 | 8.2 | IgG1 (za) | Kappa | 8.7 | 17 | 18 |
| | | | (K; DL) | | | | |
| GMCSF | 156.0 | 8.2 | IgG2 | Kappa | 8.8 | 19 | 20 |
| (3.112) | | | | | | | |
| CGRP | 159.5 | 8.3 | IgG2 | Kappa | 8.7 | 21 | 22 |
| (32H7) | | | | | | | |
| CGRP | 161.1 | 8.4 | IgG2 | Lambda | 8.6 | 23 | 24 |
| (3B6.2) | | | | | | | |
| PCSK9 | 150.0 | 9.1 | IgG2 | Kappa | 6.7 | 25 | 26 |
| (8A3.1) | | | | | | | |
| PCSK9 | 150.0 | 9.2 | IgG2 | Kappa | 6.9 | 27 | 28 |
| (492) | | | | | | | |
| CGRP | 155.2 | 9.6 | IgG2 | Lambda | 8.8 | 29 | 30 |
| Hepcidin | 147.1 | 9.9 | IgG2 | Lambda | 7.3 | 31 | 32 |
| TNFR | 157.0 | 10.0 | IgG2 | Kappa | 8.2 | 33 | 34 |
| p55) | | | | | | | |
| OX40L | 144.5 | 10.0 | IgG2 | Kappa | 8.7 | 35 | 36 |
| HGF | 155.8 | 10.6 | IgG2 | Kappa | 8.1 | 37 | 38 |
| GMCSF | 162.5 | 11.0 | IgG2 | Kappa | 8.1 | 39 | 40 |
| Glucagon R | 146.0 | 12.1 | IgG2 | Kappa | 8.4 | 41 | 42 |
| GMCSF | 144.5 | 12.1 | IgG2 | Kappa | 8.4 | 43 | 44 |
| (4.381) | | | | | | | |
| Sclerostin | 155.0 | 12.1 | IgG2 | Kappa | 7.8 | 45 | 46 |
| (13F3) | | | | | | | |
| CD-22 | 143.7 | 12.2 | IgG1 (f) | Kappa | 8.8 | 47 | 48 |
| | | | (R; EM) | | | | |
| INFgR | 154.2 | 12.2 | IgG1 (za) | Kappa | 8.8 | 49 | 50 |
| | | | (K; DL) | | | | |
| Ang2 | 151.5 | 12.4 | IgG2 | Kappa | 7.4 | 51 | 52 |
| TRAILR2 | 158.3 | 12.5 | IgG1 (f) | Kappa | 8.7 | 53 | 54 |
| | | | (R; EM) | | | | |
| EGFR | 141.7 | 14.0 | IgG2 | Kappa | 6.8 | 55 | 56 |
| IL-4R | 145.8 | 15.2 | IgG2 | Kappa | 8.6 | 57 | 58 |
| IL-15 | 149.0 | 16.3 | IgG1 (f) | Kappa | 8.8 | 59 | 60 |
| | | | (R; EM) | | | | |
| IGF1R | 159.2 | 17.3 | IgG1 (za) | Kappa | 8.6 | 61 | 62 |
| | | | (K; DL) | | | | |
| IL-17R | 150.9 | 19.1 | IgG2 | Kappa | 8.6 | 63 | 64 |
| Dkk1 | 159.4 | 19.6 | IgG2 | Kappa | 8.2 | 65 | 66 |
| (6.37.5) | | | | | | | |
| Sclerostin | 134.8 | 20.9 | IgG2 | Kappa | 7.4 | 67 | 68 |
| TSLP | 134.2 | 21.4 | IgG2 | Lambda | 7.2 | 69 | 70 |
| Dkk1 | 145.3 | 22.5 | IgG2 | Kappa | 8.2 | 71 | 72 |
| (11H10) | | | | | | | |
| PCSK9 | 145.2 | 22.8 | IgG2 | Lambda | 8.1 | 73 | 74 |
| GIPR | 150.0 | 23.0 | IgG1 (z) | Kappa | 8.1 | 75 | 76 |
| (2G10.006) | | | (K; EM) | | | | |
| Activin | 133.9 | 29.4 | IgG2 | Lambda | 7.0 | 77 | 78 |
| Sclerostin | 150.0 | 30.0 | IgG2 | Lambda | 6.7 | 79 | 80 |
| (2B8) | | | | | | | |
| Sclerostin | 141.4 | 30.4 | IgG2 | Kappa | 6.8 | 81 | 82 |
| c-fms | 146.9 | 32.1 | IgG2 | Kappa | 6.6 | 83 | 84 |
| α4β7 | 154.9 | 32.7 | IgG2 | Kappa | 6.5 | 85 | 86 |

* An exemplary concentration suitable for patient administration;
\hat{ }HC—antibody heavy chain; LC—antibody light chain.

Based on the foregoing description, it should be appreciated that the devices. systems, and methods described herein facilitate the automatic (or substantially automatic) preparation of a sample of a product containing polypeptides for glycan analysis and the automatic (or substantially automatic) performance of a glycan assay of that sample. Thus, the preparation and analysis can be performed substantially in-real time. In other words, the entire process can be performed much more quickly than presently allowed by conventional processes.

It should also be appreciated that the devices, systems, and methods described herein allow the process of preparing the sample for glycan analysis and performance of the glycan assay of that sample using the system 100 to be easily monitored, which can in turn mitigate risk and extend a production run of the product. In particular, this process can be monitored by determining, e.g., using a controller such as the controller 132 and/or manually by an operator of the system 100, whether conditions in the system 100 are optimal, i.e., whether they satisfy a pre-determined performance threshold. As illustrated in FIGS. 3D and 3E, for example, % Glycosylation values for a set of glycosylation species (in this case % M5, A1G0F, A2G0F, % HM, % Fuc, % Gal, A2G1F, and % Hybrid) can be obtained and compared against historical values for those glycosylation species collected during the method qualification to determine whether conditions in the system 100 are optimal, such that the production run can be commenced, continued, or extended. However, when it is determined that the conditions in the system 100 are not optimal, at least one cell culture component can be adjusted until the conditions in the closed system are optimal. Examples of cell culture components that can be adjusted include, but are not limited to: pH, pressure, temperature, media flow (e.g., media flow rate, media feed rate), media content (including amino acids, nutrients, sugars, buffer), gassing strategy (e.g., mix of oxygen and carbon dioxide, gas rate), agitation (e.g., agitation rate), additives (e.g., metal additives, sugar additives), additive (e.g., anti-foam) feed rate, and perfusion rate. In some cases, only one cell culture component may need to be adjusted so that the conditions in the closed system are optimal, while in other cases, multiple cell culture components may need to be adjusted. Alternatively, when it is determined that the conditions are not optimal, or when the conditions in the system 100 are not optimal even after adjusting the at least one cell culture component, the controller and/or the operator of the system 100 may shut down the system 100.

In this manner, the devices, systems, and methods described herein also mitigate risk involved in the continued operation of the system 100 when conditions are not optimal or when it is otherwise undesirable to continue operation of the system 100. In particular, risk can be mitigated by determining (e.g., calculating or obtaining) process parameter and product quality data, e.g., pH, temperature, oxygen dissolution, cell viability, cell density, titer, aggregation, charge variant, glycosylation, etc., associated with the current operation of the system 100, determining whether the process parameter and product quality data satisfy a pre-determined risk threshold (determined before operation of the system 100 by a controller such as the controller 132 and/or responsive to input from the operator of the system 100), and then determining whether to continue, cease, or adjust operation of the system 100 based upon whether the process parameter and product quality data satisfy the pre-determined risk threshold. The pre-determined risk threshold may, for example, be determined by (1) applying available process parameter data and product quality data to calculate average historical multi-variate (MV) data associated with the previous operation of the system 100 or some other similar system, and then (2) establishing the calculated average historical MV data as the threshold (the threshold can be the calculated average historical MV data itself or some value or set of values based on the calculated average historical MV data. In one example, the pre-determined risk threshold may represent an acceptable deviation (e.g., three standard deviations) from the calculated average historical MV data. Alternatively or additionally, the pre-determined risk threshold may be determined based upon input from the operator of the system 100. In some cases, the system 100 may be shut down when the process parameter and product quality data associated with the current operation of the system 100 do not satisfy (e.g., exceed) the pre-determined risk threshold. In other cases, however, the system 100 may be adjusted when the process parameter and product quality data associated with the current operation of the system 100 do not satisfy the pre-determined risk threshold or even when the process parameter and product quality data satisfy but are close to the pre-determined risk threshold. For example, and as briefly discussed above, the sampling frequency from the vessel 136 may be adjusted.

Further, the Applicant has discovered that the devices, systems, and methods described herein also allow production runs using the system 100 to be extended. Conventional processes typically allow for 32-40 day production runs, at most. However, the Applicant has found that the devices, systems, and methods described herein allow for 50-80 if not 100 population doublings, i.e., approximately 50-80 if not 100 day production runs. Thus, more product can be obtained, all while operation of the system 100 is monitored to ensure that the product satisfies quality objectives and risk is mitigated.

Preferred embodiments of this disclosure are described herein, including the best mode or modes known to the inventors for carrying out the disclosure. Although numerous examples are shown and described herein, those of skill in the art will readily understand that details of the various embodiments need not be mutually exclusive. Instead, those of skill in the art upon reading the teachings herein should be able to combine one or more features of one embodiment with one or more features of the remaining embodiments. Further, it also should be understood that the illustrated embodiments are exemplary only, and should not be taken as limiting the scope of the disclosure. All methods described herein can be performed in any suitable order unless otherwise indicated herein or otherwise clearly contradicted by context. The use of any and all examples, or exemplary language (e.g., “such as”) provided herein, is intended merely to better illuminate the aspects of the exemplary embodiment or embodiments of the disclosure, and do not pose a limitation on the scope of the disclosure. No language in the specification should be construed as indicating any non-claimed element as essential to the practice of the disclosure.

1-19. (canceled)

20. A closed system for preparing a sample in real-time for glycan analysis, the closed system comprising:
a bioreactor adapted to contain polypeptides;
a holding coil fluidly coupled to the bioreactor and arranged to receive a sample of the polypeptides from the bioreactor;
a multi-port valve fluidly coupled to and located downstream of the holding coil;
a polypeptide-binding column fluidly coupled to the multi-port valve and arranged to receive the sample from the holding coil via a first port of the multi-port valve;
a glucanase source fluidly coupled to the multi-port valve and arranged to supply glucanases to the polypeptide-binding column, such that the glucanases are infused with the polypeptides bound to the polypeptide-binding column;
a carrier solution source fluidly coupled to the multi-port valve and arranged to supply a carrier solution to the polypeptide-binding column, wherein the carrier solution is adapted to release and carry the glycans from the polypeptide-binding column;
a first pump arranged downstream of the polypeptide-binding column to direct a glycan labeling reagent toward the released glycans carried from the polypeptide-binding column, such that the glycan labeling reagent mixes with the glycans;
a reaction coil arranged downstream of the polypeptide-binding column and the first pump, the reaction coil adapted to receive the mixture comprising the glycan labeling reagent and the released glycans and to permit labeling of the released glycans;
a cooling coil arranged downstream of the reaction coil and adapted to receive the mixture from the reaction coil, the cooling coil configured to reduce a temperature of the mixture;
a glycan-binding column fluidly coupled to the cooling coil and to the multi-port valve via a second port of the multi-port valve, the glycan-binding column configured to bind the labeled glycans, the glycan-binding column adapted to receive an elution buffer solution via the second port of the multi-port valve, wherein the elution buffer solution elutes bound labeled glycans and carries the eluted labeled glycans to a glycan analysis device downstream of the glycan-binding column.

21. (canceled)

22. The system of claim 20, further comprising a vacuum apparatus arranged upstream of the reaction coil and downstream of the polypeptide-binding column and the first pump, the vacuum apparatus configured to remove gas bubbles from the mixture comprising the glycan labeling reagent and the released glycans.

23. The system of claim 20, further comprising a mixing chamber arranged downstream of the polypeptide-binding column and upstream of the reaction coil, wherein the mixing chamber facilitates an adjustment of an elution buffer component of the labeled glycans to match a starting chromatographic mobile phase condition.

24. The system of claim 20, wherein the multi-port valve comprises a 12 satellite port and a central shared port valve.

25. The system of claim 20, wherein the polypeptide-binding column is selected from the group consisting of a protein A column, a protein G column, a protein A/G column, a protein L column, an amino acid column, an avidin column, a streptavidin column, a carbohydrate bonding column, a carbohydrate column, a glutathione column, a heparin column, a hydrophobic interaction column, an immunoaffinity column, a nucleotide/coenzyme column, a specialty column, and an immobilized-metal affinity chromatography (IMAC) column.

26. The system of claim 20, wherein the glucanases are selected from the group consisting of endoglycosidases, glycosamidases, and O-glycanases, and combinations thereof.

27. The system of claim 26, wherein the endoglycosidases are selected from the group consisting of endoglycosidase D, endoglycosidase F, endoglycosidase H, endoglycosidase S, endoglycosidase M, and endoglycosidase B.

28. The system of claim 26, wherein the glycosamidases are selected from the group consisting of glycopeptidases, peptide N-glycosidases, PNGases, N-glycohydrolases, and N-glycanases.

29. The system of claim 28, wherein the PNGases is peptide: N-glycosidase F (PNGF).

30. The system of claim 26, wherein the O-glycanases are endo-GalNAc-ase D or endoGalNAc-ase A.

31. The system of claim 20, wherein the glycan labeling reagent is a fluorophore or a chromophore.

32. The system of claim 31, wherein the fluorophore is selected from the group consisting of 2-aminobenzoic acid, 8-aminonaphthalene-1,3,6-trisulfonic acid disodium salt, 8-aminonaphthalene-1,3,6-trisulfonic acid trisodium salt, and anthranilamide, 4-methoxybenzamidine.

33. The system of claim 31, wherein the chromophore comprises 3-methyl-1-phenyl-2-pyrazoline-5-one or phenylhydrazine.

34. The system of claim 20, wherein the glycan-binding column is a porous graphitic carbon column.

35. The system of claim 20, further comprising a second pump arranged upstream of the multi-port valve to pump the sample of the polypeptides from the bioreactor to the holding coil.

36. The system of claim 20, further comprising a controller communicatively coupled to the multi-port valve, the controller configured to selectively open and close the first and second ports of the multi-port valve.

37. The system of claim 20, further comprising a heating element positioned immediately adjacent the reaction coil, the heating element configured to maintain the reaction coil at a temperature of approximately 80 degrees Celsius.

38. A closed system for preparing a sample in real-time for glycan analysis, the closed system comprising:
a multi-port valve;
a holding coil upstream of the multi-port valve;
a polypeptide-binding column fluidly coupled to and downstream of a first port of the multi-port valve;
a reaction coil arranged downstream of the polypeptide-binding column;
a cooling coil arranged downstream of the reaction coil;
a glycan-binding column fluidly coupled to and downstream of the cooling coil, the glycan-binding column fluidly coupled to the multi-port valve via a second port of the multi-port valve; and
a controller communicatively coupled to the multi-port valve and comprising a memory, a processor, and logic stored on the memory and executable by the processor to:
(a) move a sample of a product containing polypeptides to the holding coil;
(b) position the multi-port valve in a first position in which the holding coil is fluidly coupled to the polypeptide-binding column via the first port of the multi-port valve, such that the sample flows to the first column, whereby the polypeptides in the sample bind to the polypeptide-binding column;
(c) when the multi-port valve is in the first position, move glucanases to the polypeptide-binding column via the holding coil to release glycans from the bound polypeptides, and then move a carrier solution to the polypeptide-binding column, via the holding coil, and through the polypeptide-binding column, thereby moving the released glycans out of the polypeptide-binding column;
(d) move the released glycans toward the reaction coil;
(e) move a glycan-labeling reagent toward the released glycans prior to reaching the reaction coil, such that the glycan-labeling reagent mixes with the released glycans;
(f) move the mixture of the released glycans and the glycan-labeling reagent to the reaction coil, whereby the glycans in the mixture are labeled;
(g) move the mixture to a cooling coil arranged downstream of the reaction coil, wherein the cooling coil reduces a temperature of the mixture;
(h) move the mixture to the glycan-binding column, whereby the labeled glycans bind to the glycan-binding column;
(i) position the multi-port valve in a second position in which the glycan-binding column is fluidly coupled to a source of elution buffer solution via the second port of the multi-port valve; and
(j) when the multi-port valve is in the second position, move an elution buffer solution from the elution buffer solution source to the glycan-binding column, and through the glycan-binding column, thereby eluting glycans bound to the glycan-binding column.

39. The system of claim 38, further comprising a vacuum apparatus arranged upstream of the reaction coil and downstream of the polypeptide-binding column and the first pump, the vacuum apparatus configured to remove gas bubbles from the mixture comprising the glycan labeling reagent and the released glycans.

40. The system of claim 38, further comprising a mixing chamber arranged downstream of the polypeptide-binding column and upstream of the reaction coil, wherein the mixing chamber facilitates an adjustment of an elution buffer component of the labeled glycans to match a starting chromatographic mobile phase condition.

41. The system of claim 38, wherein the logic is executable by the processor to move the sample from a vessel containing the sample to the holding coil.

42. system of claim 38, wherein the vessel comprises a bioreactor.

43. The system of claim 38, wherein the logic is executable by the processor to move the eluted glycans from the glycan-binding column to a glycan analysis device.

44. The system of claim 38, wherein the polypeptide-binding column is selected from a group consisting of a protein A column, a protein G column, a protein A/G column, a protein L column, an amino acid column, an avidin column, a streptavidin column, a carbohydrate bonding column, a carbohydrate column, a glutathione column, a heparin column, a hydrophobic interaction column, an immunoaffinity column, a nucleotide/coenzyme column, a specialty column, and an immobilized-metal affinity chromatography (IMAC) column.

45. The system of claim 38, wherein the glucanases are selected from the group consisting of endoglycosidases, glycosamidases, and O-glycanases, and combinations thereof.

46. The system of claim 45, wherein the endoglycosidases are selected from the group consisting of endo D, endoglycosidase F, endoglycosidase H, endo S, endo M, and endo B.

47. The system of claim 45, wherein the glycosamidases are selected from the group consisting of glycopeptidases, peptide N-glycosidases, PNGases, N-glycohydrolases, and N-glycanases.

48. The system of claim 47, wherein the PNGases is peptide: N-glycosidase F (PNGF).

49. The system of claim 45, wherein the O-glycanases are endo-GalNAc-ase D or endoGalNAc-ase A.

50. The system of claim 38, wherein the glycan-binding column is a porous graphitic carbon column.

51. The system of claim 38, wherein the glycan labeling reagent is a fluorophore or a chromophore.

52. The system of claim 51, wherein the fluorophore is selected from the group consisting of 2-aminoacridone, 8-aminonaphthalene-1,3,6-trisulfonic acid disodium salt, 8-aminonaphthalene-1,3,6-trisulfonic acid trisodium salt, and anthranilamide, 4-methoxybenzamidine.

53. The system of claim 51, wherein the chromophore comprises 3-Methyl-1-phenyl-2-pyrazoline-5-one or phenylhydrazine.

54. The system of claim 38, wherein the controller is configured to maintain the polypeptide-binding column at a temperature of approximately 38 degrees Celsius.

55. The system of claim 54, wherein the controller is configured to maintain the reaction coil at a temperature of approximately 80 degrees Celsius.

56. The system of claim 20, wherein the polypeptide of the product is a therapeutic polypeptide.

57. The system of claim 56, wherein the therapeutic polypeptide is selected from the group consisting of an antibody or antigen-binding fragment thereof, a derivative of an antibody or antibody fragment, and a fusion polypeptide.

58. The system of claim 57, wherein the antibody is selected from the group consisting of infliximab, bevacizumab, ranibizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, alemtuzumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enokizumab, enoticumab, enoticumab, ensituximab, epitumomab cituxetan, epratuzumab, erlizumab, ertumaxomab, etaracizumab, etrolizumab, exbivirumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, telimomab aritox, tenatumomab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tremelimumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox, and those antibodies shown in Table 1.

59. The system of claim 58, wherein the therapeutic polypeptide is a polypeptide selected from the group consisting of a glycoprotein, CD polypeptide, a HER receptor polypeptide, a cell adhesion polypeptide, a growth factor polypeptide, an insulin polypeptide, an insulin-related polypeptide, a coagulation polypeptide, a coagulation-related polypeptide, albumin, IgE, a blood group antigen, a colony stimulating factor, a receptor, a neurotrophic factor, an interferon, an interleukin, a viral antigen, a lipoprotein, calcitonin, glucagon, atrial natriuretic factor, lung surfactant, tumor necrosis factor-alpha and-beta, enkephalinase, mouse gonadotropin-associated peptide, DNAse, inhibin, activing, an integrin, protein A, protein D, a rheumatoid factor, an immunotoxin, a bone morphogenetic protein, a superoxide dismutase, a surface membrane polypeptide, a decay accelerating factor, an AIDS envelope, a transport polypeptide, a homing receptor, an addressin, a regulatory polypeptide, an immunoadhesin, a myostatin, a TALL polypeptide, an amyloid polypeptide, a thymic stromal lymphopoietin, a RANK ligand, a c-kit polypeptide, a TNF receptor, and an angiopoietin, and biologically active fragments, analogs or variants thereof.

60. A method of monitoring the online, real-time assay performed by the closed system of claim 20, the method comprising:
determining whether conditions in the closed system satisfy a pre-determined performance threshold; and
when it is determined that the conditions in the closed system do not satisfy the pre-determined performance threshold, adjusting at least one cell culture component until the conditions in the closed system satisfy the pre-determined performance threshold.

61. The method of claim 60, wherein adjusting at least one cell culture component comprises adjusting one or more of pH, pressure, temperature, media flow, media content, gassing strategy, agitation, additive content, additive feed rate, or perfusion rate.

62. A method of extending a production run using the closed system of claim 20, the method comprising:
determining whether conditions in the closed system satisfy a pre-determined performance threshold; and
when it is determined that the conditions in the closed system do not satisfy the pre-determined performance threshold, adjusting at least one cell culture component until the conditions in the closed system satisfy the pre-determined performance threshold.

63. The method of claim 62, wherein adjusting at least one cell culture component comprises adjusting one or more of pH, pressure, temperature, media flow, media content, gassing strategy, agitation, additive content, additive feed rate, or perfusion rate.

64. A method of mitigating risk, the method comprising:
determining process and product quality data associated with the operation of the closed system of claim 20;
determining whether the process and product quality data satisfy a pre-determined risk threshold; and
determining whether to continue operation of the closed system based upon whether the process and product quality data satisfy the pre-determined risk threshold.


SEQUENCE LISTING (86 sequences)

SEQ ID NO: 1 - protein, 239 aa, Homo sapiens
MRLPAQLLGLLMLWVPGSSGDIVMTQTPLSSPVTLGQPASISCRSSQSLVHSDGNTYLNWLQQRPGQPPRLLIYKISNRFSGVPDRFTGSGAGTDFTLKISRVEAEDVGVYTCMQVTQFPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 470 aa, Homo sapiens [sequence omitted]

SEQ ID NO: 3 - protein, 235 aa, Homo sapiens
METPAQLLFLLLLWLPDTTGEIVLTQSPGTLSLSPGDRATLSCRASQSVSSSYFAWYQQKPGQAPRLLIYGASSRATGIPDRFSGSGSGTDFTLTISRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 463 aa, Homo sapiens
MDWTWRILFLVAAATGAHSQVQLVQSGAEVKKPGASVKVSCKSSGYTFTGYYMHWVRQAPGQGLEWMGWINPNSGGTNYAQKFKGRVTMTRDTSISTAYMELSRLRSDDTAVYYCARDKWLDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 235 aa, Homo sapiens
MAWALLLLTLLTQDTGSWAQSVLTQSPSASGTPGQRVTISCSGSSSNIGSNYVYWYQQLPGAAPKLLILRNNQRPSGVPDRFSGSKSGTSASLTISGLRSEDEADYYCAAWDDSLSGWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 6 - protein, 479 aa, Homo sapiens
MDMRVPAQLLGLLLLWLRGARCEVQLVESGGGLVKPGGSLRLSCAASGFTFSNAWMSWVRQAPGKGLEWVGRIKSKTDGGTTDYTAPVKGRFTISRDDSKNTLYLQMNSLKAEDTAVYYCTTDRTGYSISWSSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 235 aa, Homo sapiens
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 467 aa, Homo sapiens
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 242 aa, Homo sapiens
MDMRVPAQLLGLLLLWLRGARCDIVMTQTPLSLPVTPGEPASISCRSSQSLLNSVDGSTNLDWYLQKPGQSPQLLIYTLSYRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 465 aa, Homo sapiens
MELGLCWVFLVAILEGVQCEVQLVESGGGLVQPGGSLRLSCAASGFTFSSYSMNWVRQAPGKGLEWVSYISSSGSSIYYADSVKGRFTISRDNAKNSLYLQMNSLRDEDTAVYYCARERYYGDTPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 233 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 463 aa, Homo sapiens
MGSTAILALLLAVLQGVCAEVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 236 aa, Homo sapiens
MDMRVPAQLLGLLLLWLRGARCDIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 467 aa, Homo sapiens
MDMRVPAQLLGLLLLWLRGARCEVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 236 aa, Homo sapiens
MDMRVLAQLLGLLLLCFPGARCDIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 466 aa, Homo sapiens
MELGLNWVFLVAILEGVHCEVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 241 aa, Homo sapiens
MDMRLPAQLLGLLMLWVPGSSGDVLMTQSPLSLPVTLGQPASISCRSSQSIVHSNGNTYLEWYLQRPGQSPKLLIYKVSNRFSGVPDRFSGSGSGTDFTLKISRVEAEDVGVYYCFQGSHVPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 470 aa, Homo sapiens
MDTLCSTLLLLTIPSWVLSQVTLKESGPALVKPTQTLTLTCTFSGFSLRTSGMGVGWIRQPPGKALEWLAHIWWDDDKSYNPSLKSQLTISKDTSKNQVVLTMTNMDPVDTATYYCARRNYYYDDYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 235 aa, Homo sapiens
METPAQLLFLLLLWLPDTTGEIVLTQSPGTLSLSPGERATLSCRASQSVSSSYFAWYQQKPGQAPRLLIYGTSSRATGIPDRFSGSGSGTDFTLTVSRLEPEDFAVYYCQQYDRSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 463 aa, Homo sapiens
MDWTWRILFLVAAATGAHSQVQLVQSGAAVKKPGASVKVSCKASGYTFTGYYIHWVRQAPGQGLEWMGWINPNSGGTNYAQKFQGRVTMTRDTSISTASMELSRLRSDDTAVYFCARDRWLDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 235 aa, Homo sapiens
METPAQLLFLLLLWLPDTTGEIVLTQSPGTLSLSPGERATLSCRASQSVSSGYLTWYQQKPGQAPRLLIYGASSRATGIPDRFSGSGSGTDFTLTISRLEPEDFAVYYCQQYGNSLSRFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 474 aa, Homo sapiens
MDMRVPAQLLGLLLLWLRGARCQVQLVESGGGVVQPGRSLRLSCAASGFTFSSYGMHWVRQAPGKGLEWVAVIWYDGSNKYYADSVKGRFIISRDKSKNTLYLQMNSLRAEDTAVYYCARAGGIAAAGLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 236 aa, Homo sapiens
MDMRVPAQLLGLLLLWLRGARCSSELTQDPTVSVALGQTVKITCQGDSLRSFYASWYQQKPGQAPVLVFYGKNNRPSGIPDRFSGSSSGNTASLTITGAQAEDEADYYCNSRDSSVYHLVLGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 24 - protein, 478 aa, Homo sapiens
MDMRVPAQLLGLLLLWLRGARCQVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARDQMSIIMLRGVFPPYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 475 aa, Homo sapiens
MDMRVPAQLLGLLLLWLRGARCEVQLVESGGGLVQPGGSLRLSCAASGFTFSSYWMSWVRQAPGKGLEWVASIKQDGSEKYYVDSVKGRFTISRDNARNSLYLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 241 aa, Homo sapiens
MDMRVPAQLLGLLLLWLRGARCDIVMTQSPLSLPVTPGEPASISCRSSQSLLHSNGYNYLDWYLQKPGQSPQLLIYLGSNRASGVPDRFSGSGSGTHLTLKISRVEAEDVGVYYCMQTLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 475 aa, Homo sapiens
MDMRVPAQLLGLLLLWLRGARCEVQLVESGGGLVQPGGSLRLSCAASGFTFSNYWMSWVRQAPGKGLEWVASIKQDGSEKYYVDSVKGRFAISRDNAKNSLFLQMNSLRAEDTAVYYCARDLVLMVYDIDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 235 aa, Homo sapiens
MTCSPLLLTLLIHCTGSWAQSVLTQPPSVSAAPGQKVTISCSGSSSNIGNNYVSWYQQLPGTAPKLLIYDNNKRPSGIPDRFSGSKSGTSTTLGITGLQTGDEADYYCGTWDSRLSAVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 30 - protein, 475 aa, Homo sapiens
MEFGLSWVFLVALLRGVQCQVQLVESGGGVVQPGRSLRLSCAASGFTFSSFGMHWVRQAPGKGLEWVAVISFDGSIKYSVDSVKGRFTISRDNSKNTLFLQMNSLRAEDTAVYYCARDRLNYYDSSGYYHYKYYGMAVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 234 aa, Homo sapiens
MDMRVPAQLLGLLLLWLRGARCYELTQPPSVSVSPGQTATITCSGDKLGERYASWYQQRPGQSPVLVIYQDIKRPSGIPERFSGSNSGNTATLTISGTQAMDEADYFCQAWYSSTNVLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 466 aa, Homo sapiens
MEFGLSWVFLVALLRGVQCQVQLVESGGGVVQPGRSLRLSCAASGFTFSSYGMHWVRQAPGKGLEWVAVIWYAESNKYYADSVKGRFTISRDNSKNTLYLQMNSLRAEDTAVYYCARAQEGIAPDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 234 aa, Homo sapiens
MGSTAILGLLLAVLQGGRAEIVLTQSPGTLSLSPGERATLSCRASQSVSSSYLAWHQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 463 aa, Homo sapiens
MGSTAILGLLLAVLQGGRAEVQLLESGGGLVQPGGSLRLSCAASGFTFSTYVMSWVRQAPGKGLEWVSSISGSGLGSYYADSVKGRFTISRDNSKNTLYLQMNSLRAEDTAVYYCAKEAHRGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 239 aa, Homo sapiens
MRLLAQLLGLLMLWVPGSSGDIVMTQTPLSSPVTLGQPASISCRSSQSLVHSDGNTYLSWLQQRPGQPPRLLIYKKFNRFSGVPDRFSGSGAGTDFTLKISRVEAEDVGVYYCMQATQIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 472 aa, Homo sapiens
MEFGLSWVFLVALLRGVQCQVQLVESGGGVVQPGRSLRLSCAASGFTFSFYGMHWVRQAPGKGLEWVAVIWYDGSNKYYADSVKGRFTISRDNSKNTLYLQMNSLRAEDTAVYYCARGGYDYVWGSYRRNSDFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 235 aa, Homo sapiens
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 465 aa, Homo sapiens
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 240 aa, Homo sapiens
MVLQTQVFISLLLWISGAYGDIVLTQSPDSLAVSLGERATINCKSSQSILYSSSNENFLTWYQQKPGQPPKLLIYWASTRESGVPDRFSGSGSGTDFTLTISSLQPEDVAVYYCQQYFSVFRTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 465 aa, Homo sapiens
MDWTWRILFLVAAATGAHSQVQLVQSGAEVKKPGASVKVSCKASGYTFTGYYIHWVRQAPGQGLEWMGWINPNSGGTNSAQKFRGRVTMTRDTSISTAYMELSRLRSDDTAVYYCAREGGYSYGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 41 - protein, 236 aa, Homo sapiens
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 473 aa, Homo sapiens
MEFGLSWVFLVALLRGVQCQVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 235 aa, Homo sapiens
METPAQLLFLLLLWLPDTTGEIVLTQSPGTLSLSPGERATLSCRASQYISNTYLAWFQQKPGQAPRLLIYGAATRATGIPDRFSGSGSGTDFTFTISRLEPEDFAVYYCQQYGSSPWTFGQGTTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 463 aa, Homo sapiens
MDWTWRILFLVAAATGAHSQVQLVQSGAEVKKPGASVKVSCKASGYTFTGYYMHWVRQAPGQGLEWMGWINPNSGGTNYAQRFRGRVTMTRDTSISTAYMELSRLRSDDTAVYYCARAPYDWTFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 45 - protein, 236 aa, Homo sapiens
MDMRVPAQLLGLLLLWLRGARCDIQMTQSPSSVSASVGDRVTITCRASQGISNWLAWYQQKPGTAPKLLIYAASSLQSGVPSRFSGSGSGTDFTLTISSLQPEDFATYYCQQANSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 469 aa, Homo sapiens
MDMRVPAQLLGLLLLWLRGARCQVQLVQSGTEVKKPGASMKVSCKASGYTFTSYYMHWVRQAPGQGLEWMGIINPSGDSTSYAQKFQGRVTMTRDTSTNTVYMELSSLRSEDTAMYYCARDVEVRGISHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 47 - protein, 238 aa, Homo sapiens
MGWSCIILFLVATATGVHSDIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 465 aa, Homo sapiens
MGWSCIILFLVATATGVHSQVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 235 aa, Homo sapiens
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 466 aa, Homo sapiens
MGSTAILALLLAVLQGVCAEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 241 aa, Homo sapiens
MDMRVPAQLLGLLLLWLRGARCDIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQGTHWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 470 aa, Homo sapiens
MDMRVPAQLLGLLLLWLRGARCEVQLVQSGGGVVQPGRSLRLSCAASGFTFSSYGMHWVRQAPGKGLEWVSYISSSGSTIYYADSVKGRFTISRDNAKNSLYLQMNSLRAEDTAVYYCARDLLDYDLLTGYGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - protein, 235 aa, Homo sapiens
METPAQLLFLLLLWLPDTTGEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 471 aa, Homo sapiens
MKHLWFFLLLVAAPRWVLSQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 236 aa, Homo sapiens
MDMRVPAQLLGLLLLWLSGARCDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 464 aa, Homo sapiens
MKHLWFFLLLVAAPRWVLSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 57 - protein, 236 aa, Homo sapiens
METPAQLLFLLLLWLPDTASEIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 460 aa, Homo sapiens
MGSTAILGLLLAVLQGVASEVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 234 aa, Homo sapiens
METPAQLLFLLLLWLPDTTGEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 467 aa, Homo sapiens
MGSTAILALLLAVLQGVCAEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 241 aa, Homo sapiens
MDMRVPAQLLGLLLLWLRGARCDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 471 aa, Homo sapiens
MDMRVPAQLLGLLLLWLRGARCQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 234 aa, Homo sapiens
MEAPAQLLFLLLLWLPDTTGEIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 461 aa, Homo sapiens
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 239 aa, Homo sapiens
MRLPAQLLGLLMLWIPGSSADIVMTQTPLSLSVTPGQPASISCKSGQSLLHSDGKTYLYWYLQKPGQPPQFLIYEVSNRFSRVPDRFSGSGSGTDFTLRISRVEAEDVGIYYCMQSIQLPWTFGQGTQVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 459 aa, Homo sapiens
MEFGLSWVFLVALLRGVQCQVQLVESGGGVVQPGRSLRLSCAASGFTFSGYGMHWVRQAPGKGLEWVAVISYDGNDKYYADSVKGRFTISRDNAKNTLYLQMNSLRAEDTAVYYCARELRVLWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 235 aa, Homo sapiens
MDMRVPAQLLGLLLLWLRGARCDIQMTQSPSSLSASVGDRVTITCRASQDISSYLNWYQQKPGKAPKLLIYSTSRLNSGVPSRFSGSGSGTDFTLTISSLQPEDFATYYCQQDIKHPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 465 aa, Homo sapiens
MDWTWRILFLVAAATGAHSEVQLVQSGAEVKKPGSSVKVSCKASGFTFTDYIMHWVRQAPGQGLEWMGYINPYNDDTEYNEKFKGRVTITADKSTSTAYMELSSLRSEDTAVYYCARSIYYYDAPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 69 - protein, 233 aa, Homo sapiens
MGSTAILGLLLAVLQGGRASYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 467 aa, Homo sapiens
MGSTAILGLLLAVLQGGRAQMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 71 - protein, 234 aa, Homo sapiens
MGVPTHLLGLLLLWITHAICDIRMTQSPASLSASLGETVNIECLASEDIYSDLAWYQQKPGKSPQLLIYNANSLQNGVPSRFSGSGSGTQYSLKINSLQSEDVATYFCQQYNNYPPTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 72 - protein, 465 aa, Homo sapiens
MDIRLSLAFLVLFIKGVQCEVQLVESGGGLVQPANSLKLSCAASGFTFSDYAMAWVRQSPKKGLEWVATIIYDGSSTYYRDSVKGRFTISRDNAKSTLYLQMDSLRSEDTATYYCATGLGIATDYFDYWGQGVLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 73 - protein, 237 aa, Homo sapiens
MDMRVPAQLLGLLLLWLRGARCESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 460 aa, Homo sapiens
MDWTWRILFLVAAATGVHSEVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 75 - protein, 234 aa, Homo sapiens
METPAQLLFLLLLWLPDTTGEIVMTQSPATLSVSPGERATLSCRASQSVSSNLAWYQQKPGQAPRLLIYGAATRATGIPARVSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 472 aa, Homo sapiens
MDMRVPAQLLGLLLLWLRGARCQVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 231 aa, Homo sapiens
MAWIPLFLGVLAYCTGSVASYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 467 aa, Homo sapiens
MDWTWSILFLVAAATGAHSQVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 79 - protein, 235 aa, Homo sapiens
MAWAPLLLTLLAHCTGSWANFMLTQPHSVSESPGKTVAISCTRNSGSIASNSVQWYQQRPGSSPTTVIFEDNQRPSGVPDRFSGSIDSSSNSASLTISGLKTEDEADYYCQSYDSNNWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 80 - protein, 470 aa, Homo sapiens
MEFGLSWVFLVALLRGVQCQVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCAREGYDYGEDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 81 - protein, 236 aa, Homo sapiens
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 468 aa, Homo sapiens
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 83 - protein, 240 aa, Homo sapiens
MVLQTQVFISLLLWISGAYGDIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 465 aa, Homo sapiens
MDWTWSILFLVAAPTGAHSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 85 - protein, 236 aa, Homo sapiens
MDMRVPAQLLGLLLLWLRGARCDIQMTQSPSSVSASVGDRVTITCRASQGISSWLAWYQQKPGKAPKLLIYGASNLESGVPSRFSGSGSGTDFTLTISSLQPEDFANYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 466 aa, Homo sapiens
MDMRVPAQLLGLLLLWLRGARCQVQLVQSGAEVKKPGASVKVSCKVSGYTLSDLSIHWVRQAPGKGLEWMGGFDPQDGETIYAQKFQGRVTMTEDTSTDTAYMELSSLKSEDTAVYYCATGSSSSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK